(19) United States
(12) Patent Application Publication
(10) Pub. No.: US 2006/0148870 A1
(43) Pub. Date: Jul. 6, 2006

(54) Process for preparing olmesartan medoxomil AT pH higher than 2.5

(76) Inventors: Lilach Hedvati, Doar Na Hefer (IL); Gideon Pilarsky, Holon (IL); Natalia Shenkar-Garcia, Petach Tiqva (IL)

Correspondence Address: KENYON & KENYON LLP, ONE BROADWAY, NEW YORK, NY, 10004, US

(21) Appl. No.: 11/217,471

(22) Filed: Sep. 2, 2005

Related U.S. Application Data
(60) Provisional application No. 60/640,183, filed on Dec. 30, 2004.

(51) Int. Cl. A61K 31/4178; C07D 405/14

(52) U.S. Cl. 514/381; 548/253

(57) ABSTRACT

The present invention provides a process for preparing olmesartan medoxomil at pH higher than 2.5.

This application claims the benefit of U.S. Provisional Patent Application Ser. No. 60/640,183 filed Dec. 30, 2004.

### FIELD OF INVENTION

The present invention relates to a process for preparing olmesartan medoxomil having reduced levels of impurities.

### BACKGROUND OF THE INVENTION

The chemical name for olmesartan medoxomil is 4-(1-hydroxy-1-methylethyl)-2-propyl-1-[[2′-(1H-tetrazol-5-yl)[1,1′-biphenyl]-4-yl]methyl]-1H-imidazole-5-carboxylic acid (5-methyl-2-oxo-1,3-dioxol-4-yl)methyl ester (Merck Index 13th ed.).

The chemical structure of olmesartan medoxomil is:

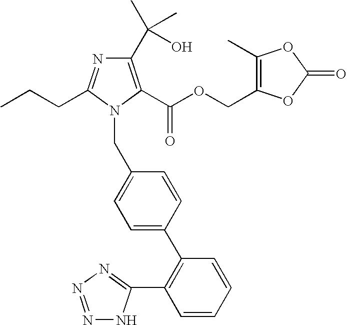

The empirical formula is C₂₉H₃₀N₆O₆.

The molecular weight is 558.58.

Olmesartan medoxomil is a prodrug that is hydrolyzed during absorption, and it is a selective AT₁ subtype angiotensin II receptor antagonist. Olmesartan medoxomil is disclosed by U.S. Pat. No. 5,616,599 to Yanagisawa et al. It is marketed as BENICAR® in film-coated tablets of 5 mg, 20 mg, and 40 mg for treatment of hypertension in a human.

The synthesis of olmesartan medoxomil (OLM-Mod) per se is illustrated as follows (see also Annu. Rep. Sankyo Res. Lab 2003, 55, 1-91):

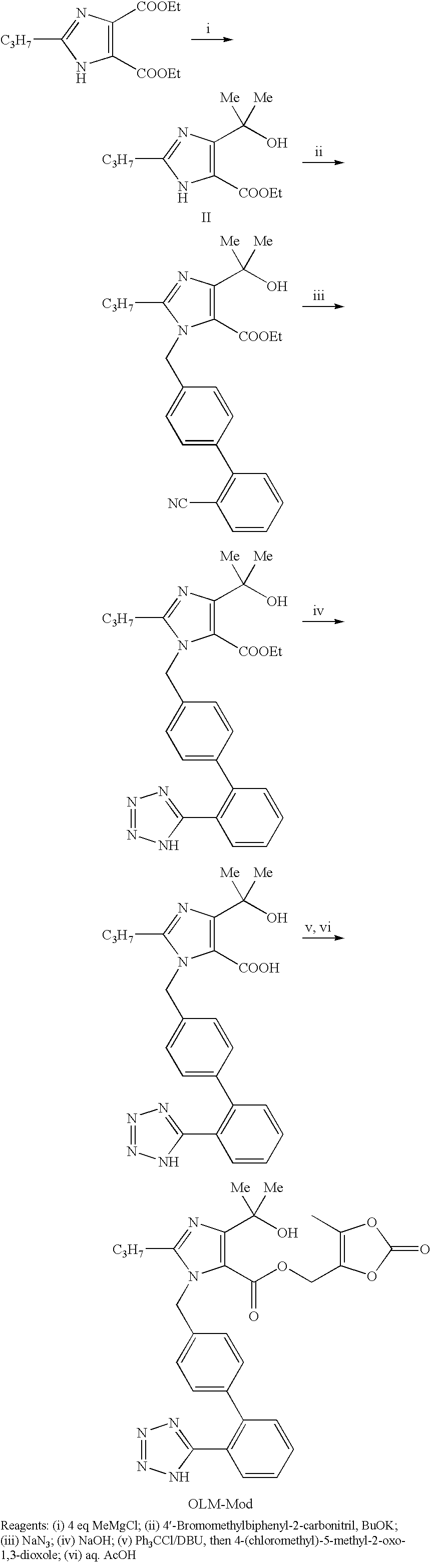

The prior art synthetic methods focus on the coupling between the substituted imidazole and the substituted biphenyl methylene bromide. Additional synthetic methods for these olmesartan medoxomil intermediates are described by: JP11302260, JP 11292851, JP 07053489, JP 06298683, U.S. Pat. No. 5,621,134, EP 838458, DE 19757995, U.S. Pat. No. 6,111,114, and U.S. Pat. No. 6214999.

Step (vi) (the deprotection step) of the prior art synthesis is illustrated as follows:

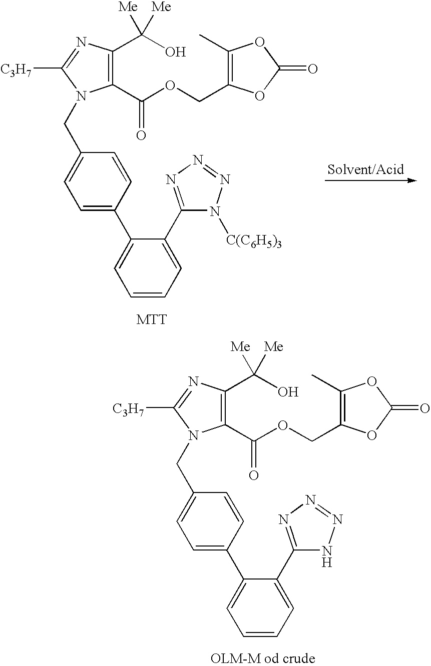

Example 61(b) of the '599 patent discloses a process for preparing crude olmesartan 10 medoxomil from a mixture of trityl olmesartan medoxomil (MTT) and aqueous acetic acid. Col. 176, lines 24-37. The deprotection step of the '599 process uses a pH lower than 2.5. Continued exposure to acidic conditions may cause decomposition of the product. Because of the acidic conditions and the presence of water, the impurity OLM-acid is also formed during the reaction by hydrolysis of the ester bond.

There is a need for improved processes for preparing olmesartan medoxomil.

### SUMMARY OF THE INVENTION

In one aspect, the present invention provides a process for preparing olmesartan medoxomil including the steps of: dissolving trityl olmesartan medoxomil in a mixture of an organic solvent, preferably acetonitrile, and water to form a first solution having a pH of at least about 2.5; and heating the first solution to obtain olmesartan medoxomil. The pH of the first solution is preferably about 3 to about 5, more preferably about 4 to about 5. The process can also include a step of adding water during the heating step.

### DETAILED DESCRIPTION OF THE INVENTION

The present invention provides a process for preparing olmesartan medoxomil including the steps of: dissolving trityl olmesartan medoxomil in a mixture of an organic solvent and water to form a first solution, wherein the first solution has a pH of at least 2.5; and heating the first solution to obtain olmesartan medoxomil. Accordingly, a process of the present invention can be illustrated as follows:

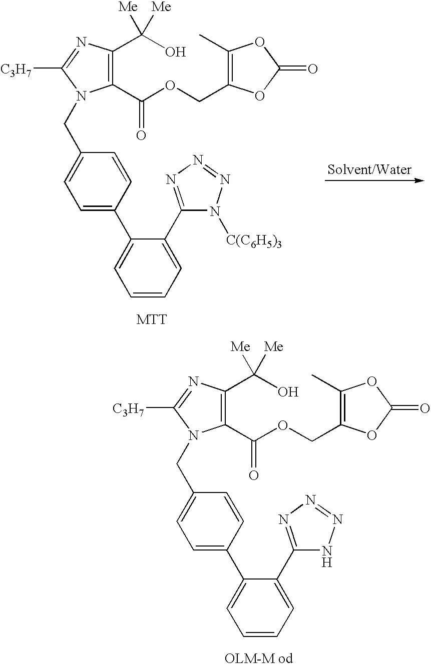

In a preferred embodiment, the pH of the first solution is about 3 to about 5, more preferably about 4 to about 5.

According to the present invention, dissolving a substance in a solvent to form a solution includes, but does not require, complete dissolution. The dissolving step also encompasses incomplete dissolution of the substance in the solvent whereby a mixture or slurry is formed.

The amount of water in the first solution depends on the organic solvent used. Preferably, the trityl olmesartan medoxomil is dissolved in a mixture of an organic solvent and about 10% to about 50% water, most preferably about 20% water.

The organic solvent of the first solution is a polar solvent, and can be protic or aprotic. The organic solvent of the first solution can be, for example, acetonitrile (ACN), iso-propyl alcohol (IPA), tert-butyl alcohol (t-BuOH), n-propyl alcohol (n-propanol), n-butyl alcohol (n-BuOH), 2-butyl alcohol (2-BuOH), iso-penthanol, dimethylamine (DMA), or dimethyl formamide (DMF). Acetonitrile is most preferred. In a preferred embodiment, the organic solvent is acetonitrile, iso-propyl alcohol, or tert-butyl alcohol, and an additional amount of water is added during the heating step to complete the reaction. When water is added, a preferred amount is an additional 1 volume of water.

The first solution is heated to a temperature of about 50° C. to about the reflux temperature of the first solution. The reflux temperature depends on the organic solvent used. With the exemplary organic solvents described above, the first solution is heated to a temperature of about 80° C. to about 110° C.

The reaction progress, e.g., the amount of trityl olmesartan medoxomil, can be measured by any method known in the art, such as, for example, HPLC, GC, TLC, NMR, and mass spectroscopy.

The first solution is preferably stirred until the amount of trityl olmesartan medoxomil is less than about 4% area by HPLC, preferably until the amount of trityl olmesartan medoxomil is less than about 2% area by HPLC. This period of time is solvent dependent. With the exemplary organic solvents described above, the reaction time is about 2.5 to about 24 hours, preferably about 2.5 to about 7 hours.

The process can further include recovering the product, olmesartan medoxomil, from the first solution by any means known in the art. Preferably, olmesartan medoxomil is recovered by evaporating the first solution to obtain a residue; dissolving the residue in a C₁₋₆ alkyl ester to form a second solution; optionally heating the second solution; cooling the second solution to precipitate olmesartan medoxomil; and recovering olmesartan medoxomil from the second solution by methods such as filtration.

C₁₋₆ alkyl esters include t-butyl methyl ester, methyl acetate, t-butyl acetate, ethyl acetate, and isopropyl acetate. Preferably, the C₁₋₆ alkyl ester is ethyl acetate.

For instance, the precipitate from the first solution can be dissolved in a small volume of the C₁₋₆ alkyl ester, e.g., 1 volume. The ester can be evaporated, and the resulting solid can be dissolved in a larger volume of the ester, e.g., 12 volumes. This C₁₋₆ alkyl ester solution can be heated, preferably to reflux; cooled, preferably to about 0° C. to about 25° C., most preferably to about 0° C.; and stirred, preferably for about 2 to about 24 hours, most preferably for about 2 hours. The final product, olmesartan medoxomil, is then filtered from the C₁₋₆ alkyl ester solution. The olmesartan medoxomil can also be washed and dried. For example, the olmesartan medoxomil can be washed with 1 volume C₁₋₆ alkyl ester and dried under vacuum at 45° C.

### EXAMPLES

### Example 1

### Comparative Example using Acetic Acid

A solution of MTT in 10 volumes of acetic acid (75%) was heated for 1.5 hrs at 60° C. until a pH of 2.21-2.23 was achieved, and the reaction was stirred until the amount of MTT was less than 2%. The mixture was evaporated to dryness. Ethyl acetate (EtOAc, 1 volume) was added to the residue and then evaporated again (twice). The resulting solid was dissolved in EtOAc (12 vol) and heated to reflux. The solution was cooled (2° C.) and stirred for 2 hrs. The product was filtered, washed (EtOAc, 1 vol), and dried on vacuum (45° C.).

### Example 2

A solution of MTT in an organic solvent and water (20%) was heated for 4-8 hrs at reflux. When the solvents were either acetonitrile (ACN), isopropyl alcohol (IPA) or t-butanol (t-BuOH), 1 volume of water was added, and the reaction was stirred until the amount of MTT was less than 2%. The mixture was evaporated to dryness. Ethyl acetate (EtOAc, 1 volume) was added to the residue and then evaporated again (twice). The resulting solid was dissolved in EtOAc (12 vol) and heated to reflux. The solution was cooled (2° C.) and stirred for 2 hrs. The product was filtered, washed (EtOAc, 1 vol), and dried on vacuum (45° C.).

Table 1 shows the process details with different organic solvents:

TABLE 1

| Total solvent | | | Time | |
|---|---|---|---|---|
| Solvent(s) | Volume | Temperature (° C.) | (hrs) | pH |
| ACN:H₂O | 5:1 + 1 | 85 | 7 | 4.89-4.3 |
| IPA:H₂O | 5:1 + 1 | 85 | 7 | 4.62-4.25 |
| t-BuOH:H₂O | 5:1 + 1 | 85 | 7 | 4.78-4.28 |
| n-propanol:H₂O | 5:1 | reflux | 2.5 | 4.3 |
| n-BuOH:H₂O | 5:1 | 110 | 2.5 | 4.41 |
| 2-BuOH:H₂O | 5:1 | 100 | 3 | 4.5 |
| iso-penthanol:H₂O | 5:1 | 100 | 3 | 5 |
| DMA:H₂O | 5:1 | 100 | 4 | 4.5 |
| DMF:H₂O | 5:1 | 100 | 4 | 4.5 |

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods.

What is claimed is:

1. A process for preparing olmesartan medoxomil comprising:
a) dissolving trityl olmesartan medoxomil in a mixture of an organic solvent and water to form a first solution, wherein the first solution has a pH of at least 2.5;
b) heating the first solution to obtain olmesartan medoxomil.

2. The process of claim 1, wherein the pH of the first solution is about 3 to about 5.

3. The process of claim 2, wherein the pH of the first solution is about 4 to about 5.

4. The process of claim 1, wherein the organic solvent is n-propyl alcohol, n-butyl alcohol, 2-butyl alcohol, iso-penthanol, dimethylamine, or dimethyl formamide.

5. The process of claim 1, wherein the organic solvent is acetonitrile, iso-propyl alcohol, or tert-butyl alcohol.

6. The process of claim 5, wherein the organic solvent is acetonitrile.

7. The process of claim 5, further comprising adding an additional amount of water to the solution during the heating step b).

8. The process of claim 7, wherein the amount of water added is about 1 volume.

9. The process of claim 1, wherein the mixture of an organic solvent and water contains about 10% to about 50% water.

10. The process of claim 9, wherein the mixture of an organic solvent and water contains about 20% water.

11. The process of claim 1, wherein the first solution is heated to a temperature of about 50° C. to about the reflux temperature of the first solution.

12. The process of claim 11, wherein the first solution is heated to a temperature of about 80° C. to about 110° C.

13. The process of claim 1, wherein step b) further comprises stirring the first solution until the amount of trityl olmesartan medoxomil is less than about 4%.

14. The process of claim 13, wherein step b) further comprises stirring the first solution until the amount of trityl olmesartan medoxomil is less than about 2%

15. The process of claim 13, wherein the stirring of the first solution is performed for about 2.5 to about 24 hours.

16. The process of claim 15, wherein the stirring of the first solution is performed for about 2.5 to about 7 hours.

17. The process of claim 1, further comprising recovering olmesartan medoxomil by evaporating the first solution to obtain a residue; dissolving the residue in a C₁₋₆ alkyl ester to form a second solution; cooling the second solution to precipitate olmesartan medoxomil; and recovering olmesartan medoxomil from the second solution.

18. The process of claim 17, wherein the C₁₋₆ alkyl ester is t-butyl methyl ester, methyl acetate, t-butyl acetate, ethyl acetate, or isopropyl acetate.

19. The process of claim 18, wherein the C₁₋₆ alkyl ester is ethyl acetate.
